(19) United States
(12) Patent Application Publication
(10) Pub. No.: US 2024/0016705 A1
(43) Pub. Date: Jan. 18, 2024

(54) COLORING SOLUTIONS FOR GUM TISSUE COLOR MATCH FOR ZIRCONIA PROSTHESIS

(71) Applicant: James R. Glidewell Dental Ceramics, Inc., Newport Beach, CA (US)

(72) Inventors: Yan Yang, Irvine, CA (US); Daniel Markowitz, Costa Mesa, CA (US); Thienphuc Nguyen, Orange, CA (US); Andrew Schissler, Garden Grove, CA (US); Sreeram Balasubramanian, Irvine, CA (US)

(73) Assignee: James R. Glidewell Dental Ceramics, Inc., Newport Beach, CA (US)

(21) Appl. No.: 18/344,001

(22) Filed: Jun. 29, 2023

Related U.S. Application Data
(60) Provisional application No. 63/358,697, filed on Jul. 6, 2022.

(51) Int. Cl. A61K 6/78; A61K 6/58; A61C 13/08; A61C 13/083

(52) U.S. Cl. CPC A61K 6/78; A61K 6/58; A61C 13/082; A61C 13/083

(57) ABSTRACT

A method that includes applying a liquid pre-coloring solution to a gum section of a zirconia dental prosthesis, wherein the liquid pre-coloring solution comprises erbium in an amount of at least 10 wt %, based on the total weight of the composition.

This application claims the benefit of and priority to U.S. Provisional Patent Appl. No. 63/358,697, filed Jul. 6, 2022, which is incorporated herein by reference in its entirety.

### BACKGROUND

Full arch dental implant supported restorations have been documented to have high success rates. The shade of the gum section plays an important role for mimicking natural gum tissue color. Conventionally, the gum section of the dental prosthesis will be pre-colored using pink color followed by gum tissue staining to match a patient's natural gum color. However, pink pre-coloring solutions currently available in the market suffer from a poor match to a patient's gum color. Numerous staining cycles (usually 3-5 cycles) after the pre-coloring are needed to produce better shade match to gum tissue color. The numerous staining, cycles extends the manuflicturing time of zirconia full arch or other zirconia prostheses with a gum tissue section, and lowers production efficiency.

### SUMMARY

Disclosed herein is a method comprising: applying a liquid pre-coloring solution to a gum section of a bisque-state zirconia dental prosthesis, wherein the liquid pre-coloring solution comprises erbium in an amount of at least 10 wt %, based on the total weight of the composition.

Disclosed herein is a method comprising:
applying an aqueous pre-coloring solution to a gum section of a bisque-state zirconia dental prosthesis, wherein the aqueous pre-coloring solution comprises erbium 30 in an amount of 10 wt % to 30 wt %, aluminum in an amount of 0 wt % to 0.3 wt %, zinc in an amount of 0 wt % to 0.5 wt %, neodymium in an amount of 0 wt % to 4 wt %, cobalt in an amount of 0 wt % to 0.2 wt %, and nickel in an amount of 0 wt % to 1.5 wt %, based on the total weight of the aqueous composition.

Disclosed herein is a method comprising:
applying an aqueous pre-coloring solution to a gum section of a bisque-state zirconia dental prosthesis, wherein the aqueous pre-coloring solution comprises erbium in an amount of 15 wt % to 27 wt %, aluminum in an amount of 0.04 wt % to 0.2 wt %, zinc in an amount of 0.05 wt % to 0.3 wt %, neodymium in an amount of 0.05 wt % to 3.6 wt %, cobalt in an amount of 0.02 wt % to 0.15 wt %, and nickel in an amount of 0.05 wt % to 1.05 wt % , based on the total weight of the aqueous composition.

Disclosed herein is a method comprising:
applying an aqueous pre-coloring solution to a gum section of a bisque-state zirconia dental prosthesis, wherein the aqueous pre-coloring solution comprises erbium in an amount of 20 wt % to 25 wt %, aluminum in an amount of 0.04 wt % to 0.06 wt %, zinc in an amount of 0.26 wt % to 0.3 wt %, neodymium in an amount of 0.05 wt % to 3.6 wt %, cobalt in an amount of 0.02 wt % to 0.15 wt %, and nickel in an amount of 0.05 wt % to 1.05 wt % , based on the total weight of the aqueous composition.

Disclosed herein is a method comprising:
applying an aqueous pre-coloring solution to a gum section of a bisque-state zirconia dental prosthesis, wherein the aqueous pre-coloring solution comprises erbium in an amount of 20 wt % to 25 wt %, aluminum in an amount of 0.08 wt % to 0.13 wt %, 25 zinc in an amount of 0.13 wt % to 0.18 wt %, neodymium in an amount of 0.05 wt % to 3.6 wt %, cobalt in an amount of 0.02 wt % to 0.15 wt %, and nickel in an amount of 0.05 wt % to 1.05 wt % , based on the total weight of the aqueous composition.

Disclosed herein is a method comprising:
applying an aqueous pre-coloring solution to a gum section of a bisque-state zirconia dental prosthesis, wherein the aqueous pre-coloring solution comprises erbium in an amount of 20 wt % to 25 wt %, aluminum in an amount of 0.17 wt % to 0.2 wt %, zinc in an amount of 0.07 wt % to 0.09 wt %, neodymium in an amount of 0.05 wt % to 3.6 wt %, cobalt in an amount of 0.02 wt % to 0.15 wt %, and nickel in an amount of 0.05 wt % to 1.05 wt % , based on the total weight of the aqueous composition.

Disclosed herein is a method comprising:
applying a liquid pre-coloring solution to a gum section of a zirconia dental prosthesis, and then sintering the pre-colored zirconia dental prosthesis, resulting in a sintered colored gum section having a Δa value of ≤15, and a ΔE value of ≤25, compared to corresponding shades contained on the BruxZirTM Gingival Shade Guide (Glidewell Laboratories, Newport Beach, California).

Disclosed herein is a method comprising:
applying a liquid pre-coloring solution to a gum section of a zirconia dental prosthesis, resulting in a colored gum section having an increase of least 10 in the “a” value in the L*a*b color space compared to an untreated zirconia substrate.

Disclosed herein is an aqueous composition comprising erbium in an amount of 15 wt % to 27 wt %, aluminum in an amount of 0.04 wt % to 0.2 wt %, zinc in an amount of 0.05 wt % to 0.3 wt %, neodymium in an amount of 0.05 wt % to 3.6 wt %, cobalt in an amount of 0.02 wt % to 0.15 wt %, and nickel in an amount of 0.05 wt % to 1.05 wt %, based on the total weight of the aqueous composition.

Disclosed herein is an aqueous composition comprising erbium in an amount of 20 wt % to 25 wt %, aluminum in an amount of 0.04 wt % to 0.06 wt %, zinc in an amount of 0.26 wt % to 0.3 wt %, neodymium in an amount of 0.05 wt % to 3.6 wt %, cobalt in an amount of 0.02 wt % to 0.15 wt %, and nickel in an amount of 0.05 wt % to 1.05 wt %, based on the total weight of the aqueous composition.

Disclosed herein is an aqueous composition comprising erbium in an amount of 20 wt % to 25 wt %, aluminum in an amount of 0.08 wt % to 0.13 wt %, zinc in an amount of 0.13 wt % to 0.18 wt %, neodymium in an amount of 0.05 wt % to 3.6 wt %, cobalt in an amount of 0.02 wt % to 0.15 wt %, and nickel in an amount of 0.05 wt % to 1.05 wt %, based on the total weight of the aqueous composition.

Disclosed herein is an aqueous composition comprising erbium in an amount of 20 wt % to 25 wt %, aluminum in an amount of 0.17 wt % to 0.2 wt %, zinc in an amount of 0.07 wt % to 0.09 wt %, neodymium in an amount of 0.05 wt % to 3.6 wt %, cobalt in an amount of 0.02 wt % to 0.15 wt %, and nickel in an amount of 0.05 wt % to 1.05 wt % , based on the total weight of the aqueous composition.

The foregoing will become more apparent from the following detailed description, which proceeds with reference to the accompanying figures.

### BRIEF DESCRIPTION OF THE DRAWINGS

FIG. 1. Section of a full arch used for L*a*b measurement using the SpectroShade Micro II imaging spectrophotometer.

FIG. 2. CIELAB Color Space.

FIG. 3. Planar view of the CIELAB Color Space.

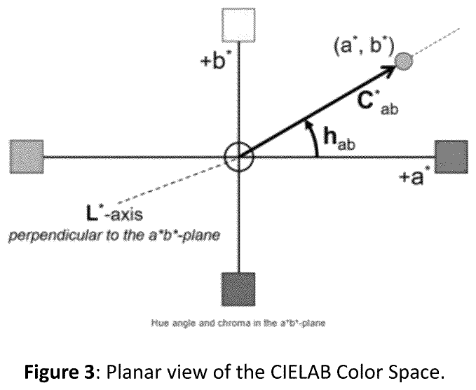

FIG. 4. BruxZirTM Gingival Shade Guide (Glidewell Laboratories, Newport Beach, California).

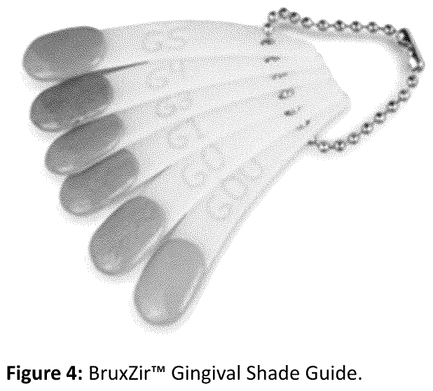

### DETAILED DESCRIPTION

Disclosed herein are pre-coloring liquid compositions for coloring a zirconia green body or bisque body to provide a gum tissue color for dental prosthesis such as full-arch implants. After drying and sintering, the resulting zirconia full-arch implant and other zirconia prostheses exhibit a better color match to gum tissue color. The compositions and methods disclosed herein shorten manufacturing time, improve working efficiency, and produce better esthetic appearances.

Dental prostheses that include a gum section are typically available in a variety of gum shades. Thus, the pre-coloring compositions disclosed herein can have a variety of components (both in specific materials and amounts) that are tailored for a specific gum shade.

In certain embodiments, the composition includes erbium in an amount of at least 10 wt %, more particularly at least 15 wt %, and most particularly at least 20 wt %, based on the total weight of the composition.

In certain embodiments, the composition includes erbium and zinc.

In certain embodiments, the composition includes erbium and aluminum.

In certain embodiments, the composition includes erbium, zinc, and aluminum.

In certain embodiments, the composition also includes cobalt and/or neodymium.

In certain embodiments, the composition also includes nickel.

In certain embodiments, the composition includes erbium, zinc, aluminum, cobalt, neodymium, and nickel.

In certain embodiments, the only color-imparting elements in the composition are erbium, zinc, aluminum, cobalt, neodymium, and nickel.

In particular embodiments, erbium is present in an amount of 10 wt % to 30 wt %, or 15 wt % to 27 wt %, more particularly 20 wt % to 25 wt %, based on the total weight of the composition.

In particular embodiments, zinc is present in an amount of at least 0.05 wt %, more particularly at least 0.07 wt %, based on the total weight of the composition. In particular embodiments, zinc is present in an amount of 0 wt % to 0.5 wt %, more particularly 0.05 wt % to 0.3 wt %, more particularly 0.26 wt % to 0.3 wt %, and 0.13 wt % to 0.18 wt %, and 0.07 wt % to 0.09 wt % based on the total weight of the composition.

In particular embodiments, aluminum is present in an amount of not more than 0.5 wt %, based on the total weight of the composition. In particular embodiments, aluminum is present in amount of 0 wt % to 0.5 wt %, more particularly in amount of 0.04 wt % to 0.2 wt %, more particularly in the amount of 0.04 wt % to 0.06 wt %, and 0.08 wt % to 0.13 wt %, and 0.17 wt % to 0.2 wt % based on the total weight of the composition.

In particular embodiments, nickel is present in an amount of 0 wt % to 1.5 wt %, more particularly 0.05 wt % to 1 wt % based on the total weight of the composition.

In particular embodiments, cobalt is present in an amount of 0 wt % to 0.2 wt %, more particularly 0.02 wt % to 0.15 wt % based on the total weight of the composition.

In particular embodiments, neodymium is present in an amount of 0 wt % to 5 wt %, more particularly 0.05 wt % to 3.7 wt %, based on the total weight of the composition.

The erbium, zinc, aluminum, nickel, cobalt, and neodymium may be provided from a salt or an oxide thereof Illustrative salts include acetate, oxalate, sulfate, carbonate, halide (e.g., chloride or fluoride), nitrate, phosphate or citrate. In certain embodiments, the component is a hydrate of the salt.

In one embodiment, the erbium, zinc, aluminum, nickel, cobalt, and neodymium is provided in the form of a metallic salt that is soluble in an aqueous composition. The erbium, zinc, aluminum, nickel, cobalt, and neodymium may be added to the composition in the form of a solid or a liquid.

Illustrative ingredients include erbium nitrate pentahydrate (e.g., Er(NO₃)₃·5H₂O), erbium nitrate hexahydrate (e.g., Er(NO₃)₃·6H₂O), zinc nitrate hexahydrate (e.g., Zn(NO₃)₂·6H₂O), cobalt nitrate hexahydrate (e.g., Co(NO₃)₂·6H₂O), cobalt chloride hexahydrate (CoCl₂·6H₂O), aluminum chloride hexahydrate (e.g, AlCl₃·6H₂O), nickel nitrate hexahydrate (e.g., Ni(NO₃)₂·6H₂O), and neodymium nitrate hexahydrate (e.g., Nd(NO₃)₃·6H2O).

The composition can be an aqueous composition or a non-aqueous composition, particularly an aqueous solution. The composition can be prepared by mixing the erbium, zinc, aluminum, nickel, cobalt, and/or neodymium salts or oxides into water at room temperature.

The composition may include a color application indicator component such as a food dye. The color application indicator component enables visualization of the composition coverage on the dental prosthesis by the person applying the composition.

The composition may be applied by techniques such as painting by brushing, or by dipping, or dripping, or infiltrating liquid coloring compositions onto/into the dental prosthesis. Compositions may be applied by known techniques for distributing liquid compositions onto ceramic surfaces, including coating with a marker or felt-tip pen that is loaded with the liquid mixture, or by use of a sponge.

Prior to the application of the composition, bisque stage dental prostheses may be unshaded or shaded, having the color of natural zirconia materials. Applying at least one coating of at least one composition imparts a pre-coloring of the gum tissue section which enables achieving a dentally acceptable gum color after sintering, glazing, and staining.

In certain embodiments, only one or two stain cycles are necessary after applying the compositions disclosed herein. Three to five stain cycles (1.5 hours per cycle) are required with conventional pre-coloring compositions to reach a target shade. Thus, the presently disclosed compositions can save 1.5 to 6 hours per arch.

In certain embodiments, applying the composition to the gum section of a bisque-state zirconia dental prosthesis, and then sintering the pre-colored zirconia dental prosthesis, results in a sintered shaded gum section having a Δa value of ≤15, more particularly Δa value of ≤10, more particularly a Δa value of ≤8, and a ΔE value of ≤25, more particularly a ΔE value of ≤15, more particularly a ΔE value of ≤10 compared to the BruxZir™ Gingival Shade Guide. A lower Δa and ΔE value indicate better shade match between treated samples and the comparison reference. In particular embodiments, after applying the pre-coloring composition to a gum section, the dental prosthesis is sintered at 1400° C. to 1700° C., more particularly 1500° C. to 1600° C., for 5 minutes to 300 minutes, more particularly 10 to 150 minutes.

In certain embodiments, applying the composition to a gum section of a zirconia dental prosthesis results in a shaded gum section having an increase of at least 10 in the “a” value (Δa), more particularly an increase of at least 14 in the “a” value, more particularly an increase of at least 18 in the “a” value in the L*a*b color space compared to an untreated zirconia substrate. Higher Δa here indicates providing more reddish color to the zirconia substrate materials to better match the hue of natural gum tissue.

The pre-coloring compositions disclosed herein can be applied to any gum section of a dental prosthesis. In certain embodiments, the dental prosthesis is a full-arch implant, or a partial-arch implant, or other single or bridge dental restorations with a gum part. For example, the arch implant may be a 100% solid monolithic zirconia implant such as the BruxZir® implant prosthesis available from Glidewell.

### Sample Preparation Method:

Zirconia ceramic material may comprise a mixture of unstabilized zirconia and stabilized zirconia ceramic materials. The term stabilized zirconia ceramic herein includes fully stabilized and partially stabilized zirconia. Specific examples include zirconia with no yttria, or yttria-stabilized zirconia including, but not limited to, commercially available yttria-stabilized zirconia, for example, from Tosoh USA, such as Tosoh TZ-3YS and Tosoh TZ-PX430. The calculated amount of yttria (e.g., yttria mol %) in zirconia ceramic material may vary from ‘nominal’ values implied by commercial nomenclature (e.g. 3YS). The mol% yttria in zirconia ceramic material may be calculated, for example, based on compositional information received from manufacturer certification.

Dental prosthetic shapes may be formed as green bodies or bisqued state bodies. Green body manufacturing methods may include dry forming processes, such as uniaxial pressing and cold isostatic pressing, and wet forming processes, including but not limited to, pressure-casting, slip-casting, filter pressing, and centrifugal casting methods. A green body manufacturing method such as a slip-casting process, may include the process steps of selecting starting materials; mixing and comminuting the starting materials to form a slurry; and casting the slurry to form a desired green body form, such as the shape of a milling blocks. Methods for making zirconia dental prosthesis materials suitable for use herein may be found in commonly owned patents and patent publications, including U.S. Pat. Nos. 9,434,651, 9,790,129, and U.S. Pat. Pub. 2018/0235847, the subject matter of each is hereby incorporated by reference in its entirety.

Yttria-stabilized zirconia ceramic materials used as starting materials to form millable blocks may, optionally, include a small amount of alumina (aluminum oxide, Al₂O₃) as an additive. For example, some commercially available yttria-stabilized zirconia ceramic material include alumina at concentrations of from 0 wt % to 2 wt %, or from 0 wt % to 0.25 wt %, such as 0.1 wt %, relative to the zirconia material. Other optional additives of the ceramic starting material may include coloring agents to obtain shaded zirconia ceramic powder that may be formed by, for example, casting or pressing into shaded ceramic blocks that have a dentally acceptable shade or pre-shade upon sintering.

Dispersants used to form ceramic suspensions or ceramic slurries to form green bodies by slip-casting manufacturing methods such as those described herein, function by promoting the dispersion and/or stability of the slurry and/or decreasing the viscosity of the slurry. Dispersion and deagglomeration may occur through electrostatic, electrosteric, or steric stabilization. Examples of suitable dispersants include nitric acid, hydrochloric acid, citric acid, diammonium citrate, triammonium citrate, polycitrate, polyethyleneimine, polyacrylic acid, polymethacrylic acid, polymethacrylate, polyethylene glycols, polyvinyl alcohol, polyvinyl pyrillidone, carbonic acid, and various polymers and salts thereof. These materials may be either purchased commercially, or prepared by known techniques. Specific examples of commercially available dispersants include Darvan® 821-A ammonium polyacrylate dispersing agent commercially available from Vanderbilt Minerals, LLC; Dolapix™ CE 64 organic dispersing agent and Dolapix™ PC 75 synthetic polyelectrolyte dispersing agent commercially available from Zschimmer & Schwarz GmbH; and Duramax™ D 3005 ceramic dispersant commercially available from Dow Chemical Company.

Zirconia ceramic and dispersant starting materials added to deionized water may be mixed to obtain a slurry. Slurries may be subjected to a comminution process for mixing, deagglomerating and/or reducing particle size of zirconia ceramic powder particles. Comminution may be performed using one or more milling process, such as attritor milling, horizontal bead milling, ultrasonic milling, or other milling or comminution process, such as high shear mixing or ultra-high shear mixing capable of reducing zirconia ceramic powder particle sizes described herein.

In one embodiment, a zirconia ceramic slurry may undergo comminution by a horizontal bead milling process. Media may comprise zirconia-based beads, for example, having a diameter of 0.4 mm. A suspension or slurry having a zirconia ceramic solids loading of about 60 wt % to about 80 wt % and a dispersant concentration from 0.002 gram dispersant/gram zirconia ceramic powder to 0.01 gram dispersant/gram zirconia ceramic powder, may be used to prepare the zirconia ceramic slurry. Milling processes may include, for example, a flow rate of 1 kg to 10 kg zirconia ceramic powder/hour, such as, approximately 6 kg zirconia ceramic powder/hour where, for example, approximately 6 kg of zirconia ceramic material is milled for approximately one hour, at a mill speed of approximately 1500 rpm to 3500 rpm, for example, approximately 2000 rpm.

In some embodiments, where commercially available zirconia ceramic is used as starting materials to prepare the ceramic slurry, the measured median particle size, or particle size distribution at D₍₅₀₎ may be about 150 nm to 600 nm, or greater than 600 nm, which includes agglomerations of particles of crystallites having a crystallite size of about 20 nm to 40 nm. As used herein, the term “measured particle size” refers to measurements obtained by a Brookhaven Instruments Corp. X-ray disk centrifuge analyzer. By processes described herein, an initial particle size distribution at, for example, a D₍₅₀₎ of about 200 nm to 600 nm, or greater than 600 nm, may be reduced to provide a zirconia ceramic material contained in a slurry having a median particle size where D₍₅₀₎ is from 100 nm to 600 nm, such as, wherein D₍₅₀₎ is from 150 nm to 350 nm, or from 220 nm to 320 nm or wherein D(50) is from 250 nm to 300 nm. In some embodiments, after comminution processes ceramic slurries comprise particle size distributions wherein D₍₁₀₎ is from 100 nm to 250 nm, or D₍₁₀₎ is from 120 nm to 220 nm, or D₍₁₀₎ is from 120 nm to 200 nm, and D₍₉₀₎ of zirconia particles is less than 800 nm, or D₍₉₀₎ is in the range of 250 nm to 425 nm.

By processes described herein, zirconia ceramic material may comprise an initial median particle size, for example, a D₍₅₀₎ of less than 400 nm, which upon comminution may provide a slurry comprising a zirconia ceramic material having a median particle size where D₍₅₀₎ is from 100 nm to 350 nm, such as, wherein D₍₅₀₎ is from 80 nm to 280 nm. Yttria-stabilized zirconia ceramic material comprising mixtures of two or more yttria stabilized zirconia ceramic materials each having different initial median particle sizes, may be comminuted as a mixture in a slurry by the processes described herein. Reduced particle sizes and/or narrow ranges of comminuted zirconia ceramic material, in combination with the dispersants describe above, may provide cast parts with a higher density and smaller pores that form sintered bodies having higher translucency and/or strength than those obtained by way of conventional pressing and slip-casting processes.

Zirconia ceramic slurries may be cast into a desired shape, such as a solid block, disk, near net shape, or other shape. Ceramic slurries may be poured into a porous mold (e.g., plaster of Paris or other porous/filtration media) having the desired shape, and cast, for example, under the force of capillary action, vacuum, pressure, or a combination thereof (for example, by methods provided in US 2013/0313738, which is hereby incorporated by reference in its entirety). Green bodies may form a desired shape as water contained in the slurry is absorbed/filtered through the porous media. Excess slurry material, if any remaining, may be poured off the green body. Green bodies removed from molds may dry, for example, at room temperature in a controlled, low humidity environment. Dental milling blanks may be cast, for example, as a solid block, disk or near-net-shape, having dimensions suitable for use in milling or grinding single unit or multi-unit restorations, such as crowns, veneers, bridges, partial or full-arch dentures, and the like.

Manufacturing processes described herein may provide green bodies having relative densities pR greater than 48%, such as from 52% to 65% relative density, or such as from 56% to 62% relative density. As used herein, the term “relative density” (ρR) refers to the ratio of the measured density pm of a sample (g/cm³) to the theoretical density ρT (3 YSZ—6.083 g/mL; 5 YSZ—6.037 g/mL; 7 YSZ—5.991 g/mL).

Green bodies may be partially consolidated to obtain bisqued bodies by a heating step. Bisquing methods include heating or firing green bodies, such as green bodies in the shape of blocks to obtain, for example, porous bisqued blocks. In some embodiments, relative densities of bisque blocks do not increase more than 5% over the green body density. In some embodiments, the ceramic bodies are bisque heated so that the difference between the relative densities of the bisque body and the green body is 3% or less. Resulting bisqued bodies may be fully dried and have strength sufficient to withstand packaging, shipping, and milling, and in some embodiments, have a hardness value of less than or equal to 0.9 GPa, when tested by the hardness test method described herein. Bisque firing steps may include heating the green body at an oven temperature of from 800° C. to 1100° C. for a holding period of about 0.25 hours to 3 hours, or about 0.25 hours to 24 hours, or by other known bisquing techniques. In some embodiments, bisque processes comprise heating green bodies in an oven heated at an oven temperature of 900° C. to 1000° C. for 30 minutes to 5 hours.

Processes described herein may provide a bisqued body having a relative density ρR greater than or equal to 48%, such as from 48% to 62%, or from 54% to 60% Bisqued bodies may have a porosity of less than or equal to 45%, such as from 35% to 45%, or from 38% to 42%, or from 38% to 41%. As used herein, the term “porosity”, expressed as percent porosity above, is calculated as: percent porosity =1 — percent relative density. A dental block for producing a dental prosthesis includes a zirconia bisqued body having a density of between 56% to 65% of theoretical density and having a porosity of between 35% and 44%, such as between 38% and 41%.

In some embodiments, the median pore size of bisque bodies is less than 200 nm, or less than 150 nm, less than 100 nm, such as from 30 nm to 150 nm, or from 30 nm to 80 nm, or from 35 nm to 40 nm, or from 40 nm to 80 nm, or from 40 nm to 70 nm, or from 45 nm to 75 nm, or from 45 nm to 50 nm, or from 50 nm to 80 nm, or from 50 nm to 75 nm, or from 55 nm to 80 nm, or from 55 nm to 75 nm, when measured according to the methods described herein. As used herein, the term “median pore diameter” refers to the pore diameter measurements obtained from a bisqued body via mercury intrusion performed with an Autopore V porosimeter from Micromeritics Instrument Corp.

Conventional subtractive processes, such as milling or machining processes known to those skilled in the art, may be used to shape a bisqued zirconia ceramic body or milling block into a pre-sintered dental restoration. For dental applications, a pre-sintered restoration may include a dental restoration such as a crown, a multi-unit bridge, an inlay or onlay, a veneer, a full or partial denture, or other dental restoration. For example, bisque stage blocks milled to form bisque-stage dental restorations having anatomical facial surface features including an incisal edge or biting surface, anatomical dental grooves and cusps, and are sintered to densify the bisque-stage restoration into the final dental restoration that may permanently installed in the mouth of a patient. In alternative embodiments, bisque-stage zirconia ceramic bodies are shaped into near-net-shape blocks having generic sizes and shapes that are sintered to theoretical density prior to machining into a final patient-specific dental restoration. The sintered near-net-shape bodies may be prepared having a shape and/or size that is suitable for range of similarly sized and shaped final restoration products.

Dental prostheses may be shaped from porous, pre-sintered blocks by conventional subtractive processes, such as milling or machining processes known to those skilled in the art. The blocks may be shaped in a crown, a multi-unit bridge, an inlay or onlay, a veneer, a full or partial denture, or other dental prosthesis.

After treating bisque stage dental prostheses by applying one or more liquid coloring compositions as disclosed herein, the bisque stage bodies may be “fully sintered” under atmospheric pressure to a density that is at least 98% of the theoretical density of a sintered body. Sintering may occur at oven temperatures in the range of 1200° C. to 1900° C., or 1400° C. to 1600° C., or 1450° C. to 1580° C. Hold times (dwell times) at a temperature within a sintering temperature range may be from 1 minute to 48 hours, such as from 10 minutes to 5 hours, or from 30 minutes to 4 hours, or from 1 hour to 4 hours, or from 1 hour to 3 hours, or from 2 hours to 2.5 hours. Other sintering processes include multi-step sintering processes described in commonly owned U.S. Pat. Pub. 2019/0127284, filed Oct. 31, 2018, hereby incorporated herein by reference in its entirety. Multi-step sintering processes may comprise one or more temperature gradients within a sintering temperature range, with each gradient having the same or different ramp rates, reaching oven temperatures at or above 1200° C., such as from 1200° C. to 1900° C. Multi-step sintering methods may optionally having no hold time within a sintering temperature range, or one hold time or multiple hold times at or above 1200° C. Multi-step sintering processes may have multiple temperature peaks at or above 1200° C., and at least one temperature steps that is between 25° C. to 600° C. lower, or between 50° C. to 400° C. lower, than a preceding or subsequent temperature peak. Hold times at temperature peaks may be between 0 minutes and 30 minutes, and a lower temperature step between two temperature peaks may have a hold time between 2 minutes and 5 hours.

### Measurement Method:

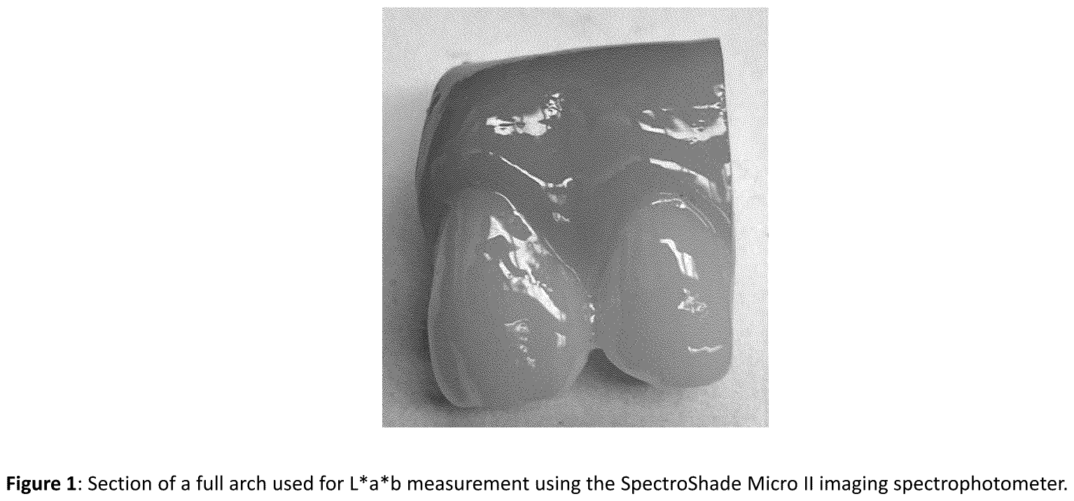

Spectral image data of the labial face of the gingiva of sectioned full arch restorations was collected using a SpectroShade Micro II imaging spectrophotometer (see FIG. 1). Prior to collecting spectral image data, the SpectroShade Micro II was calibrated in accordance with built-in calibration instructions provided with the instrument - using the white and green tiles on the docking base provided with the unit.

Restorations were cleaned with isopropyl alcohol and imaged over a dark background (the AC/DC switching adaptor supplied with the SpectroShade Micro II; MEAN WELL ENTERPRISES, GS40A15-P1M). A small dot of wax was used to support each full arch restoration upon the dark background such that the greatest proportion of the labial face of the gingiva was approximately level with the dark background surface and exposed for spectral imaging. The SpectroShade Micro II (with mouthpiece attached) was then aligned by hand and used to capture a spectral image measurement file for each sample.

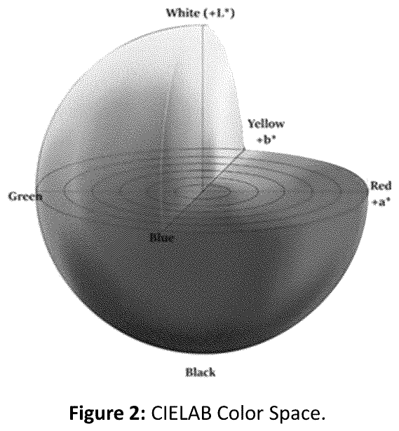

The CIELAB Color Space is the tool used to evaluate shade and color match between samples (see FIG. 2). The two planar axes are denoted as “a” and “b”. The “a” axis represents the colors red and green, with a positive value being associated with red, and a negative value being associated with green. The “b” axis represents the colors yellow and blue, with a positive value being associated with yellow, and a negative value being associated with blue. The “L” axis represents how light or dark the sample is. A high “L” value is associated with lighter (white) shades, and a low value is associated with darker (black) shades. The values of “a” and “b” range from −100 to +100 respectively, and the values of “L” range from 0 to 100

When comparing the color of two samples, the ΔE value can be calculated using each sample's respective L*a*b values. It is, in essence, a distance formula between two points in the L*a*b Color Space. If the ΔE value is ≤2, the color difference between the two samples is indiscernible. The ΔE value is calculated using the following equation:

ΔE=\sqrt{(b2−b1)²+(a2−a1)²+(L2−L1)²}

Further information can be gathered from viewing the CIELAB Color Space in a planar view (excluding “L” values). In FIG. 2, h_{ab} denotes the hue and c*_{ab} denotes the chroma. The hue, measured as an angle of rotation in the 2-D plane, describes the pigment without accounting for how “light” or “dark” the color is. The chroma is a measure of the color saturation. Hue and chroma are calculated by the following equations:

$$Hue = arctan ( \frac{b}{a} )$$

$$Chroma = \sqrt{{a}^{2} + {b}^{2}}$$

It can be noted that while a=b, increasing the “a” and “b” values will not change the hue but cause a rise in the chroma value.

The L*a*b values collected from the SpectroShade were then compared to the BruxZir™ Gingival Shade Guide to evaluate the overall shade match between them.

Δa, ΔHue, and ΔChroma values were calculated by measuring the absolute value of the difference between the invention values and the Shade Guide values.

Δa=a_{Invention}−a_{Shade Guide}

ΔHue=Hue_{Invention}−Hue_{Shade Guide}

ΔChroma=Chroma_{Invention}−Chroma_{Shade Guide}

In the case of comparing inventive samples with the Shade Guide, smaller A values are an indication of better shade match.

### EXAMPLES

Solutions were made by dissolving metal salts in water. The compositions of the invented solutions with the weight percentage (wt %) of metal elements to the total solution weight are listed in Table 1:

TABLE 1
Metal Element Weight Percentages Table

| Solution # | Er | Ni | Nd | Al | Zn | Co |
|---|---|---|---|---|---|---|
| Sol. 1 | 25.00% | | | 0.18% | 0.27% | |
| Sol. 2 | 24.41% | | | 0.19% | 0.28% | |
| Sol. 3 | 23.85% | 0.23% | | | | 0.04% |
| Sol. 4 | 24.11% | 0.28% | | 0.09% | 0.14% | 0.06% |
| Sol. 5 | 22.96% | 0.66% | 2.16% | | | 0.10% |
| Sol. 6 | 23.78% | 0.34% | 1.68% | | | 0.07% |
| Sol. 7 | 22.11% | 0.07% | 0.84% | | | |
| Sol. 8 | 23.17% | 0.11% | 1.96% | | | |
| Sol. 9 | 23.85% | 0.23% | | | | 0.04% |
| Sol. 10 | 20.05% | 0.09% | 1.82% | | | |
| Sol. 11 | 23.91% | 0.71% | 0.58% | | | 0.11% |
| Sol. 12 | 22.16% | 0.26% | | 0.10% | 0.15% | 0.08% |
| Sol. 13 | 25.72% | | | | | |
| Sol. 14 | 25.15% | | | | | |
| Sol. 15 | 24.48% | 0.29% | | | | 0.06% |

TABLE 2
ΔE of 4.7 mol % Y2O3 stabilized ZrO2 samples treated with the
invention solutions compared with BruxZir ™ Gingival Shade Guide

| Sol # | ΔE |
|---|---|
| Sol. 1 | 18.7 |
| Sol. 2 | 22.3 |
| Sol. 4 | 16.1 |
| Sol. 13 | 26.3 |
| Sol. 14 | 30.3 |
| Sol. 15 | 15.2 |

TABLE 3
Sintering program for Y₂O₃ stabilized ZrO₂ dental materials

| | t1 | T1 | t2 | T2 | t2 | T3 | t4 | T4 | t5 | T5 |
|---|---|---|---|---|---|---|---|---|---|---|
| | (min) | (° C.) | (min) | (° C.) | (min) | (° C.) | (min) | (° C.) | (min) | (° C.) |
| Prog#1 | 78 | 1200 | 60 | 1200 | 50 | 1300 | 28 | 1580 | 150 | 1580 |
| Prog#2 | 78 | 1200 | 60 | 1200 | 50 | 1300 | 25 | 1450 | 1 | 1200 |

| | t6 | T6 | t7 | T7 | t8 | T8 | t9 | T9 | t10 | T10 |
|---|---|---|---|---|---|---|---|---|---|---|
| | (min) | (° C.) | (min) | (° C.) | (min) | (° C.) | (min) | (° C.) | (min) | (° C.) |
| Prog#1 | | | | | | | | | | |
| Prog#2 | 90 | 1200 | 18 | 1475 | 5 | 1475 | 8 | 1550 | 10 | 1550 |

TABLE 4
L*a*b, Hue, and Chroma Values of the BruxZir ™ Gingival Shade
Guide, Untreated 3 mol % Y2O3 stabilized ZrO2, and
Untreated 4.7 mol % Y2O3 stabilized ZrO2
BruxZir ™ Gingival Shade Guide L*a*b Values
and Untreated Zirconia L*a*b Values

| Shade | L | a | b | Hue | Chroma |
|---|---|---|---|---|---|
| G00 | 62 | 22 | 6 | 0.27 | 23.08 |
| G0 | 60 | 23 | 10 | 0.42 | 25.15 |
| G1 | 53 | 20 | 10 | 0.47 | 22.25 |
| G3 | 54 | 19 | 6 | 0.33 | 19.80 |
| G4 | 42 | 22 | 16 | 0.62 | 27.13 |
| G5 | 48 | 21 | 7 | 0.31 | 22.11 |
| 3Y Untreated | 90 | −1 | 1 | −0.35 | 1.59 |
| 4.7Y Untreated | 85 | −3 | 0 | 0.00 | 3.00 |

TABLE 5
Raw L*a*b Values and Shade Guide Comparison Values of treated 3 mol
% Y₂O₃ stabilized ZrO₂ and treated 4.7 mol % Y₂O₃ stabilized ZrO₂

| Solution # | Y₂O₃ mol % | L | a | b | Hue | Chroma | ΔL | Δa | Δb | Δ_{HUE} | Δ_{CHROMA} | ΔE |
|---|---|---|---|---|---|---|---|---|---|---|---|---|
| 1 | 3 | 72 | 11 | 2 | 0.18 | 10.72 | −10 | 12 | 4 | 0.09 | 12.36 | 16 |
| 2 | 3 | 67 | 10 | 2 | 0.22 | 10.02 | −7 | 13 | 8 | 0.20 | 15.12 | 17 |
| 3 | 3 | 75 | 15 | 9 | 0.57 | 17.40 | −22 | 5 | 1 | −0.10 | 4.85 | 23 |
| 4 | 3 | 65 | 9 | 2 | 0.23 | 9.11 | −11 | 10 | 4 | 0.11 | 10.69 | 15 |
| 5 | 3 | 44 | 13 | 7 | 0.49 | 14.48 | −2 | 9 | 9 | 0.13 | 12.65 | 13 |
| 6 | 3 | 57 | 14 | 3 | 0.20 | 14.43 | −10 | 7 | 4 | 0.11 | 7.67 | 13 |
| 1 | 4.7 | 72 | 10 | −3 | −0.34 | 10.46 | −10 | 12 | 10 | 0.61 | 12.62 | 19 |
| 2 | 4.7 | 70 | 8 | −3 | −0.37 | 8.96 | −10 | 15 | 13 | 0.79 | 16.18 | 22 |
| 4 | 4.7 | 72 | 13 | 9 | 0.60 | 15.78 | −19 | 7 | 1 | −0.14 | 6.47 | 20 |
| 4 | 4.7 | 62 | 9 | −3 | −0.34 | 9.06 | −8 | 10 | 10 | 0.68 | 10.74 | 16 |
| 5 | 4.7 | 51 | 16 | 9 | 0.52 | 18.10 | −9 | 6 | 7 | 0.10 | 9.03 | 13 |
| 6 | 4.7 | 60 | 14 | 1 | 0.05 | 14.08 | −12 | 7 | 6 | 0.26 | 8.03 | 15 |
| 7 | 4.7 | 72 | 14 | 1 | 0.07 | 14.19 | 10 | −8 | −5 | −0.20 | −8.89 | 14 |
| 8 | 4.7 | 63 | 15 | 5 | 0.34 | 15.80 | 4 | −8 | −5 | −0.08 | −9.34 | 10 |
| 9 | 4.7 | 61 | 15 | 6 | 0.36 | 16.43 | 8 | −5 | −4 | −0.10 | −5.82 | 10 |
| 10 | 4.7 | 57 | 11 | 2 | 0.19 | 11.07 | 3 | −8 | −4 | −0.14 | −8.73 | 10 |
| 11 | 4.7 | 44 | 16 | 10 | 0.57 | 18.81 | 2 | −6 | −6 | −0.05 | −8.32 | 9 |
| 12 | 4.7 | 55 | 15 | 6 | 0.40 | 16.77 | 8 | −6 | 0 | 0.09 | −5.34 | 10 |

TABLE 6
Increase in the “a” value of samples treated with invented
solutions compared to untreated samples

| | Y₂O₃ | Increase in “a” from | |
|---|---|---|---|
| | mol % | Untreated Substrate | ΔL* |
| Sol. 1 | 3 | 12 | 18 |
| Sol. 2 | 3 | 11 | 23 |
| Sol. 3 | 3 | 16 | 14 |
| Sol. 4 | 3 | 10 | 25 |
| Sol. 5 | 3 | 14 | 46 |
| Sol. 6 | 3 | 16 | 32 |
| Sol. 1 | 4.7 | 19 | 1 |
| Sol. 2 | 4.7 | 21 | 9 |
| Sol. 3 | 4.7 | 18 | 16 |
| Sol. 4 | 4.7 | 19 | 21 |
| Sol. 5 | 4.7 | 18 | 31 |
| Sol. 6 | 4.7 | 17 | 26 |

In view of the many possible embodiments to which the principles of the disclosed invention may be applied, it should be recognized that the illustrated embodiments are only preferred examples of the invention and should not be taken as limiting the scope of the invention.

What is claimed is:

1. A method comprising:
applying a liquid pre-coloring solution to a gum section of a zirconia dental prosthesis, wherein the liquid pre-coloring solution comprises erbium in an amount of at least 10 wt %, based on the total weight of the composition.

2. The method of claim 1, wherein the solution further comprises aluminum.

3. The method of claim 2, wherein the solution further comprises zinc.

4. The method of claim 3, wherein the solution further comprises neodymium.

5. The method of claim 4, wherein the solution further comprises cobalt.

6. The method of claim 5, wherein the solution further comprises nickel. 7 The method of claim 1, wherein the solution is an aqueous solution.

8. The method of claim 1, wherein the erbium is present in an amount of 10 wt % to 30 wt %, based on the total weight of the composition.

9. The method of claim 1, wherein the erbium is from erbium nitrate.

10. The method of claim 2, wherein the aluminum is present in an amount of 0.04 wt % to 0.2 wt %, based on the total weight of the composition.

11. The method of claim 3, wherein the zinc is present in an amount of 0.05 wt % to 0.3 wt %, based on the total weight of the composition.

12. The method of claim 4, wherein the neodymium is present in an amount of 0.05 wt % to 3.6 wt %, based on the total weight of the composition.

13. The method of claim 5, wherein the cobalt is present in an amount of 0.02 wt % to 0.15 wt %, based on the total weight of the composition.

14. The method of claim 6, wherein the nickel is present in an amount of 0.05 wt % to 1.05 wt %, based on the total weight of the composition.

15. A method comprising:
applying an aqueous pre-coloring solution to a gum section of a bisque-state zirconia dental prosthesis, wherein the aqueous pre-coloring solution comprises erbium in an amount of 10 wt % to 30 wt %, aluminum in an amount of 0 wt % to 0.3 wt %, zinc in an amount of 0 wt % to 0.5 wt %, neodymium in an amount of 0 wt % to 4 wt %, cobalt in an amount of 0 wt % to 0.2 wt %, and nickel in an amount of 0 wt % to 1.5 wt %, based on the total weight of the aqueous composition.

16. The method of claim 15, wherein the aqueous pre-coloring solution comprises erbium in an amount of 15 wt % to 27 wt %, aluminum in an amount of 0.04 wt % to 0.2 wt %, zinc in an amount of 0.05 wt % to 0.3 wt %, neodymium in an amount of 0.05 wt % to 3.6 wt %, cobalt in an amount of 0.02 wt % to 0.15 wt %, and nickel in an amount of 0.05 wt % to 1.05 wt %, based on the total weight of the aqueous composition.

17. The method of claim 16, wherein the only color-imparting elements in the solution are erbium, zinc, aluminum, cobalt, neodymium, and nickel.

18. The method of claim 15, further comprising sintering the pre-colored zirconia dental prosthesis.

19. The method of claim 18, wherein the sintered colored gum section has a Δa value of ≤15, and a ΔE value of ≤25, compared to BruxZir™ Gingival Shade Guide.

20. The method of claim 18, wherein the sintered colored gum section has an increase of least 10 in the “a” value in the L*a*b color space compared to an untreated zirconia substrate.

21. The method of claim 16, further comprising sintering the pre-colored zirconia dental prosthesis, and then staining the colored gum section in only one or two staining cycles.

22. A method comprising:
applying a liquid pre-coloring solution to a gum section of a zirconia dental prosthesis, and then sintering the pre-colored zirconia dental prosthesis, resulting in a sintered colored gum section having a Δa value of ≤15, and a ΔE value of ≤25, 20 compared to BruxZir™ Gingival Shade Guide.
